(19) United States
(12) Patent Application Publication
(10) Pub. No.: US 2011/0230748 A1
(43) Pub. Date: Sep. 22, 2011

(54) METHOD AND APPARATUS FOR MEASURING HEART RATE

(76) Inventor: Seppo Säynäjäkangas, Kempele (FI)

(21) Appl. No.: 12/999,869

(22) PCT Filed: Jun. 15, 2009
(86) PCT No.: PCT/FI09/50519

§ 371 Date: Jun. 13, 2011

(30) Foreign Application Priority Data
Jun. 19, 2008 (FI) 20085615

(51) Int. Cl. A61B 5/0408

(52) U.S. Cl. 600/390

(57) ABSTRACT

The main principle of the invention is that measurement of the heart rate occurs by wireline with the fixed ECG electrodes according to FIG. 1. The electronics that are unnecessary for the wireless receiver are removed from what is in itself a prior-art pulse counter and also the wireless pulse transmitter is omitted and replaced with fixed ECG electrodes, which are connected in a fixed manner to the pulse counter (1).

### FIELD OF THE INVENTION

The invention relates to a method and an apparatus for measuring heart rate.

More particularly the invention relates to a method and an apparatus for measuring heart rate with a pulse counter fixable to the arm.

### PRIOR ART

In world-class sports, sports training and rehabilitation it is important to measure the heart rate reliably without interrupting the performance and without the performance interfering with the measurement. Various wireless measuring apparatuses based on radio waves and capacitive phenomena are known, in which e.g. a receiver kept on the wrist and a transmitter fixed to a suitable part of the body are used.

FI patent publication 68374 also discloses a wireless telemetric measuring arrangement for measuring heart rate, wherein telemetric data transfer occurs using a magnetic near-field by controlling magnetic windings in the transmitter unit with a heart rate signal or ECG signal expressed from the body and amplified, the directional magnetic field produced by which windings is essentially multidimensional, and by expressing the aforementioned magnetic field in the receiver with a winding construction possessing at least a partially corresponding directional pattern of the multidimensional magnetic field. According to the method the ECG signal or heart rate of a person is measured from a suitable part of the body in a separate transmitter apparatus and this signal is amplified in a signal amplifier. With the ECG signal or heart rate signal, amplified to a suitable voltage level, via a power amplifier three magnetic windings placed in a perpendicular direction to each other are controlled with gated alternating current signals according to the heart rate. This alternating current signal controlled via the windings produces a magnetic so-called near field in the environs of the transmitter apparatus simultaneously in a manner characteristic to each winding. Thus an easily measurable 3-dimensional and almost circularly symmetrical magnetic field is produced in the environs of the transmitter from the combined effect of the windings.

Three separate winding elements situated at right angles with respect to each other are disposed in a corresponding manner in the receiver for expressing the magnetic field produced around the transmitter.

A telemetric transmitter apparatus of heart rate used on the breast and a receiver apparatus of heart rate used on the wrist are typically used in the solution according to this patent. The ECG signal of a person is expressed with the ECG electrodes disposed in the transmitter apparatus and it is converted electronically in the transmitter apparatus into an alternating current signal for supply the windings. The alternating current signal produces corresponding magnetic fields in the environs of each winding at the same time as it passes via the windings. Correspondingly, the three windings installed at right angles to each other in the receiver apparatus measure the magnetic fields in the environs of the receiver apparatus.

Transmitter units and receiver units are needed in prior-art apparatuses for transmitting measuring signals wirelessly. They consume a relatively large amount of power, and both units require their own power source. Additionally, the electronics of a receiver apparatus used on the wrist is relatively complex, because it must be able to process the data transfer signals used in wireless data transfer. This makes prior-art apparatuses relatively large, complex and expensive.

### SUMMARY OF THE INVENTION

The purpose of this invention is to achieve a new type of method and apparatus for measuring heart rate.

The invention is a method and an apparatus for the continuous measurement of heart rate by wireline in the way that is presented in detail in the independent claims below.

With the method according to the invention a low power consumption is achieved, because no transmitter apparatuses or receiver apparatuses that consume electrical power are needed. In addition, the signal is received directly in the pulse counter without complex signal processing, and no battery or other power source is needed in the separate fixed electrode.

The apparatus according to the invention is extremely simple and operationally reliable, and also inexpensive in price.

### SHORT DESCRIPTION OF THE DRAWINGS

In the following, the invention will be described in more detail with reference to the attached drawing, wherein

FIG. 1 presents an apparatus according to the invention fixed the wrist and to the arm of a person, and

FIG. 2 presents a cross section of a pulse counter unit as viewed from the side.

### DETAILED DESCRIPTION OF A PREFERRED EMBODIMENT

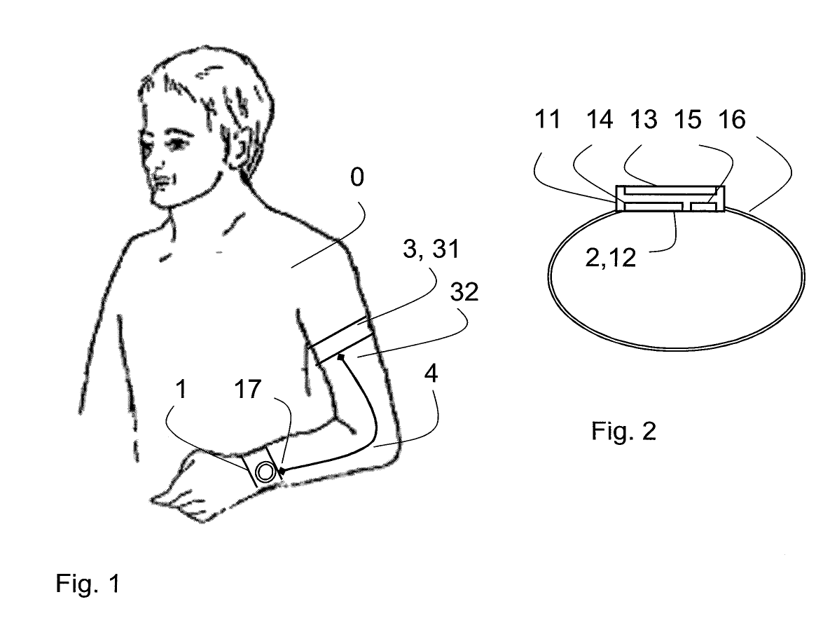

FIG. 1 presents an apparatus according to the invention for the continuous measurement of the heart rate of a person 0 by wireline. The apparatus according to the invention comprises a pulse counter unit 1, which comprises a fixed ECG electrode base 2 (FIG. 2), a fixed ECG electrode unit 3 on the arm or on the sleeve of a damp shirt, and a fixed connection conductor 4 between the unit 1 and the unit 3.

The pulse counter unit comprises a frame 11 made of a metal that conducts electricity, e.g. steel, which also includes copper, which frame thus also comprises a base 2 situated against the wrist, which base forms the second electrode 12, a display 13, and control electronics 14 and a power source 15 inside the frame. The base electrode is connected galvanically to the control electronics to form the second electrode of the circuit. In addition, the unit comprises a wristband 16 for fixing it to the wrist and a connector 17 for the connection conductor.

The fixed electrode unit, connected in a fixed manner (galvanically) to the pulse counter unit by means of the connection lead, comprises an elastic electrode band loop 31, made of e.g. a conductive rubber and of relatively large area, that can be fitted around the arm as well as a connector 32 for connecting it to the conductor 4.

The apparatus according to the invention operates as follows:

The invention is a method and an apparatus for the continuous measurement of heart rate by wireline. The method and the apparatus according to it measure the heart rate from one arm when one ECG measuring electrode is positioned on the wrist, e.g. on the base of the pulse counter, and the other fixed ECG electrode is positioned above the wrist e.g. on the arm.

The measurement is performed by measuring the ECG potential produced between the ECG electrodes, i.e. the low-voltage potential of the heart.

This low-voltage signal is amplified into a pulse beat density in the electronics part such that a pulse signal identifying the pulse frequency of the heart rate that is adequately powerful is produced, which is then converted into the numerical format to present on the display of the meter of the pulse of the person.

It must be noted that the new invention does not use a wireless approach, as prior-art pulse counters do, but instead the pulse is measured with fixed ECG electrodes.

It is obvious to the person skilled in the art that the different embodiments of the invention are not limited solely to the embodiment described above, but that they may be varied within the scope of the claims presented below. Also a damp sleeve and/or other part of the shirt can if necessary be used alongside the fixed electrode band.

1. Method for measuring heart rate with a portable pulse counter apparatus, characterized in that the heart rate is measured by wireline such
that two fixed ECG electrodes (12, 31) fixed to the body, preferably kept on the same arm, are used for the pulse measurement, the heart rate receivable from which ECG electrodes is amplified into a pulse frequency signal of the heart rate and is controlled to be presented on the display of the apparatus.

2. Method according to claim 1, characterized in that one of the fixed ECG electrodes forms the base of the pulse counter and the other fixed ECG electrode forms an elastic, fixed, band-like electrode to be fitted around a part of the body, such as the arm, and if necessary also a damp sleeve and/or other damp part of the shirt.

3. Portable pulse counter apparatus, comprising a measuring unit (1), for measuring heart rate, characterized in that the apparatus is a wireline apparatus for measuring heart rate with a wire such that the apparatus comprises two electrodes fitted to the body in such a way that
two fixed ECG electrodes (22, 31) fixed to the body, preferably fitted to the same arm, are used for the measurement of heart rate, of which at least one ECG electrode can be connected to the measuring unit with a connection conductor,
the heart rate receivable from which ECG electrodes is amplified into a pulse frequency signal of the heart rate and can be controlled to be presented on the display of the apparatus.

4. Apparatus according to claim 3, characterized in that one of the fixed ECG electrodes forms the base of the pulse counter and the other fixed ECG electrode forms an elastic, fixed, band-like electrode to be fitted around a part of the body, such as the arm, and if necessary also a damp sleeve and/or other damp part of the shirt.

5. Apparatus according to claim 3, characterized in that the apparatus comprises a separate electrode unit and a separate pulse counter unit, which comprises a frame (21), which frame comprises a base (22), made of a material that conducts electricity, to be fitted against the wrist, which base forms the second electrode, a display (23), and control electronics (24) and a power source (25) inside the frame, which base electrode is connected to the control electronics to form the second electrode of the circuit.

6. Apparatus according to claim 5, characterized in that the pulse counter unit comprises a wristband (26) for fixing it to the wrist and a connector (27) for the connection lead

7. Apparatus according to claim 3, characterized in that the electrode unit comprises an elastic electrode band (31) that at least partly conducts electricity, e.g. is made of conductive rubber, and can be fitted around the arm as well as a connector (32) for connecting it to the connection conductor (4).
